(19) United States
(12) Patent Application Publication
(10) Pub. No.: US 2017/0055545 A1
(43) Pub. Date: Mar. 2, 2017

(54) SYSTEM FOR AND METHOD OF CONVERTING AGRICULTURAL WASTE TO PROBIOTIC ANIMAL FEED

(71) Applicant: Lee Tech LLC, San Jose, CA (US)

(72) Inventor: Chie Ying Lee, San Jose, CA (US)

(73) Assignee: Lee Tech LLC

(21) Appl. No.: 15/345,336

(22) Filed: Nov. 7, 2016

Related U.S. Application Data
(63) Continuation-in-part of application No. 14/265,301, filed on Apr. 29, 2014.
(60) Provisional application No. 61/937,995, filed on Feb. 10, 2014.

(51) Int. Cl. A23K 20/189; A61K 35/747; A61K 36/064; A61K 36/062; A23K 10/18; C12P 7/56; C12M 1/33; B02C 23/08; A23K 10/37; A23K 10/12; A61K 35/745; A61K 9/00

(52) U.S. Cl. CPC A23K 20/189; A61K 35/745; A61K 35/747; A61K 36/064; A61K 36/062; A61K 9/0056; C12P 7/56; C12M 45/02; B02C 23/08; A23K 10/37; A23K 10/12; A23K 10/18; C12P 39/00

(57) ABSTRACT

Methods of and devices for producing probiotic animal feed are provided. The method includes adding probiotic microorganisms or enzymes, providing an environmental condition suitable for the growth of the probiotic microorganisms, such as providing abundant sources of carbon, controlling temperature, and controlling pH values, in a fermenting process.

### CROSS-REFERENCE TO RELATED APPLICATION(S)

This application is a Continuation-in-Part patent application of co-pending U.S. patent application Ser. No. 14/265,301, filed Apr. 29, 2014, published as U.S. Patent Application Publication No. 2015-0223493, and titled, “A SYSTEM FOR AND METHOD FOR CONVERTING AGRICULTURAL WASTE TO ANIMAL FEED AND OTHER VALUABLE RAW MATERIALS,” which claims priority to the U.S. Provisional Patent Application Ser. No. 61/937,995, filed Feb. 10, 2014 and titled, “A SYSTEM FOR AND METHOD FOR CONVERTING AGRICULTURAL WASTE TO ANIMAL FEED AND OTHER VALUABLE RAW MATERIALS,” and which are both hereby incorporated by reference in their entirety for all purposes.

### FIELD OF THE INVENTION

The present invention relates to the field of animal feed. More specifically, the present invention relates to a system for and method of processing plant waste for making probiotic animal feed.

### BACKGROUND OF THE INVENTION

The world's population continues to rapidly increase creating dire food shortages due to the limited land on earth. This can be attributed to the tendency of consuming more animal protein than plant protein. It takes several pounds of plant protein to produce one pound of animal protein. For example, it takes an average of 8 pounds of plant protein to feed a cow to produce one pound of cow protein, 4 pounds of plant protein to feed a pig to produce one pound of pig protein, 3 pounds of plant protein to feed a chicken to produce one pound of chicken protein, and 1.5 pounds of plant protein to feed a fish to produce one pound of fish protein.

The plant wastes are from various sources. For example, many agricultural wastes are produced by all agricultural crops during the harvest. These include a) grain crops (such as rice, wheat, and corn, which have (1) stalks and straws and (2) hull and cobs); b) vine and leaf crops (such as peas and soybeans, which have (1) vines and leaves and (2) pods); and c) root crops (such as cassava and potato, which have (1) vines and leaves and 2) tubes. The crop waste in the field contains several times of weight compared to the actual crop produce and has low protein content (3 to 12% protein) with less nutrient value. As a result, it has very limited use as animal feed (mainly for cows and horses). It is normally turned over into ground as soil conditioner and fertilizer. Recycling agricultural waste is a sustainable manner, which avoids current burning methods that create unhealthy air pollution.

Further, many food process plants also produce waste during the food processing steps, such as rice/wheat bran, vegetables that cannot be used for human consumption, cassava/potato skins, and orange peels from orange juice plants. These wastes consist of about 5% to 10% of crops used by humans and contain higher protein and valuable nutrients which can be used as part of animal feed.

Furthermore, farmers invest their time, energy, and resources to grow crops that are sold on the open market for supermarket distribution or further processing. When produce is harvested, the roots, shells, branches, leaves, or stems are cut. Texture and flavor properties of these remnant parts are deemed undesirable for human consumption and ultimately discarded. Recognizing that plant waste contains minerals such as nitrogen, potassium, and calcium which are vital for the soil to grow a new batch of crops, farmers use their plant waste as fertilizer. Some of the plant wastes containing higher level of vitamins, mineral and protein that are used as part of animal feed have existed and remained for hundreds of years in the area with the lack of animal feed resource.

These agricultural wastes can be valuable for human and animal consumption. The proteins, hydrocarbons, oils, antibiotics, minerals, and antioxidants total content weight in this agricultural waste is more than those in crops that are consumed by humans and animals. The majority of those valued resources, however, are not wholly used by humans and animals because the low percentage level protein content with a very high cellulose content, which is not suitable for human and animal consumption. In fact, the discarded outer layer or shell of plant waste often possesses more nutrients than the inner body sold as produce.

Currently, there are many chemicals being used in vegetable farms and antibiotics in animal farms which are known hazardous to human health.

### SUMMARY OF THE INVENTION

In an aspect, a method of making probiotic animal feed comprises adding one or more probiotic substances to a fermenting and generating a probiotic animal feed. In some embodiments, the method further comprises breaking cell wall of an agriculture substance before fermenting. In other embodiments, the method further comprises separating a liquid portion from a solid portion, wherein the liquid portion is sent to the fermenting. In some other embodiments, the probiotic substances comprise microorganisms. In some embodiments, the probiotic substances comprise enzymes of microorganisms. In other embodiments, the probiotic substances comprise enzymes. In some other embodiments, the method further comprises converting hydrocarbons to organic acids. In some embodiments, the method further comprises co-fermenting with an engineered Bacillus subtilis and engineered Lactobacillus acidophilus.

In another aspect, a method of making probiotic animal feed comprises providing an environment suitable for the proliferation of probiotic microorganisms and generating probiotic animal feed. In some embodiments, the method further comprises increasing the number of probiotic microorganisms. In other embodiments, the method further comprises a fiber and liquid separating, which generates a fiber portion and a liquid portion. In some other embodiments, the method further comprises adjusting a pH value of the liquid portion to a range between 5.5 and 7. In some embodiments, the method further comprises precipitating proteins and nutrients by performing the pH adjusting.

In another aspect, a method of making probiotic animal feed comprises collecting an agriculture substance, breaking cell walls of the agriculture substance, separating a liquid portion from a solid portion, adding microorganisms or enzymes to the liquid portion, and forming a probiotic animal feed. In some embodiments, the method further comprises fermenting the liquid portion. In other embodiments, the method further comprises adjusting a pH value of the liquid portion. In some other embodiments, the method further comprises recovering proteins, oil, antioxidants, organic antibiotics, animal nutrients, or a combination thereof. In some embodiments, the method further comprises separating oil and protein in the liquid portion. In other embodiments, the method further comprises fermenting after the separating oil and protein in the liquid portion. In some other embodiments, the method further comprises recovering protein between the fermenting and the separating oil and protein in the liquid portion. In some embodiments, the method further comprises fermenting between the separating a liquid portion from a solid portion and the separating oil and protein in the liquid portion.

In another aspect, a probiotic animal feed making system comprises a cell wall breaking device, a fermentor, and an amount of probiotic substances in the fermentor. In some embodiments, the probiotic substances comprise an amount of added microorganisms, bioengineered microorganisms, natural microorganisms, and enzymes. In other embodiments, the system further comprises organic acids. In some embodiments, the organic acids are at a level sufficient to inhibit bacteria that are harmful to animal, human, or both. In other embodiments, the organic acids comprise lactic acid, proprionic acid, benzoic acid, butyric acid, or a combination thereof. In some embodiments, the organic acids form organic anti-microbial substances.

In some embodiments, nutrients (proteins, oils, hydrocarbons, antioxidants, organic antibiotics, minerals, and vitamins) are extracted from discarded plant waste (roots, skins, seeds, stalks, leaves, and stems) using grinding and separation processes in accordance with some embodiments of the present invention. The system of the present invention is able to run at steady production state and extract valuable materials from the plant waste for making animal feed, including cellulosic materials for compost, organic fertilizer, raw materials for the paper industry, and alcohol production. All the plants/plant wastes described in the present specification are able to be used as a source of the raw material.

In some embodiments, the extraction method of the present invention uses a variety of materials including edible agricultural products, byproducts and wastes of agricultural products and plant inedible wastes. The method breaks the cell walls of the materials and releases valuable ingredients, such as protein, oil, hydrocarbon, organic antibiotics, vitamins, minerals and antioxidants. Next, separation steps are performed to separate the cellulosic material in a solid form from valuable animal nitrite compounds in a liquid slurry form. The liquid slurry with valuable compounds is further separated into protein, oil, starch, antioxidant, inorganic components (which are able to be used as animal feed) and mineral (which is able to be used as fertilize). The vitamins and other valuable chemicals are able to be optionally separated. The liquid slurry contains a lot of hydrocarbon (such as oligosaccharides, starch, and fine fiber which contain immense potential energy), which can be processed and used as a carbon source to grow probiotics or other beneficial microorganisms to produce antibiotic-free and nutritional animal feed.

In some embodiments, bacteria exhibiting cellulase, hemicellulases and/or protease activities are used to digest soluble cellulose and hemicelluloses and convert them into nutrients to populate probiotic. For example, Bacillus sp. are a group of bacteria well-known to produce cellulase and some hemicellulases. Cellulase can break cellulose into glucose, which is one of the most efficient carbon sources to grow probiotics. Hemicellulose like xylan can be broken down to five-carbon sugar (xylose) by xylanase, which also make xylan become bio-accessible. The processes of the present invention open up more carbon source to probiotics to use and grow exponentially. As they grow, probiotics consume the carbon sources and turn the carbon sources into lactic acid or other organic acids, which are known to benefit animal and human health. Protease can break large molecule proteins into amino acids and makes it easily-absorbed by animals, which cannot digest proteins easily or animals, like chicken, having a short digestion and absorption period.

In an aspect, a method of making animal feed comprises collecting an agriculture substance, breaking cell walls of the agriculture substance, removing a cellulose substance, and forming food having a content suitable for animal consumption. In some embodiments, the method comprises recovering oil. In other embodiments, the method further comprises coagulating protein. In some other embodiments, the coagulating protein comprises heating to a temperature higher than 160 F. In some embodiments, the coagulating protein comprises adjusting a pH value to be in the range between 3-6. In some embodiments, the agriculture substance comprises a plant waste. In other embodiments, the plant waste comprises roots, skins, seed, stalks, leaves, or a combination thereof. In some other embodiments, the method further comprises making a slurry by adding water to the agriculture substance. In some embodiments, the slurry has a moisture content of 70%-90%. In other embodiments, the method further comprises releases protein, oil, starch, antioxidant, nutrient, or a combination thereof by breaking cell walls. In some embodiments, the process of breaking cell walls is performed by using a milling device. In other embodiments, the milling device comprises a hammer mill, a disc mill, a pin mill, a disintegrator, a simple blender type mill, or a combination thereof. In some other embodiments, the method further comprises increasing a yield of protein recovery by using a screw press. In some embodiments, the food comprises protein, hydrocarbon, antioxidant, organic antibiotic, mineral, and vitamin. In some embodiments, the food comprises proteins, hydrocarbons, antioxidants, organic antibiotic, minerals, probiotic and vitamins. In other embodiments, the method further comprises forming animal drinking water containing minerals and substantially free of protein. In some other embodiments, the cellulose substance is treated to produce a raw material for paper industry, alcohol, animal bedding, compose, organic fertilizer, or organic packing material.

In another aspect, a system for agriculture waster converting comprises a cell wall breaking device, a cellulose separating device, an oil recovering device configured to recover oil released at the cell wall breaking device, a protein coagulating device, and a protein recovering device configured to recover a coagulated protein at the protein coagulating device. In some embodiments, the cell wall breaking device comprises a hammer mill, a disc mill, a pin mill, a disintegrator, a simple blender type mill, or a combination thereof. In other embodiments, the system further comprises a separation device. In some other embodiments, the separation device comprises a pressure screen, a paddle screen, a vibration screen, a screen centrifuge, a press, or a combination thereof.

In another aspect, a method of converting agriculture waste comprises gathering an amount of agriculture waste, mixing an amount of water with the agriculture waste to form a first slurry, breaking cell walls of the agriculture waste in the slurry using a milling device, such that proteins, hydrocarbons, antioxidants, organic antibiotics, minerals, and vitamins are released, removing cellulose from the slurry using a screen separating device, coagulating the proteins, and forming animal food.

In another aspect, a process comprises breaking plant cell wall, converting soluble fibers to animal-available nutrients, and increasing the amount of probiotics and the organic acid content in the animal feed. The main components of the cell walls comprise cellulose (a polysaccharide of β(1→4) linked D-glucose) and hemicellulose. Hemicellulose is made from various monosaccharide like xylose, mannose, galactose, rhamnose and arabinose. Hemicellulose binds cellulose together to form fiber bundles to increase strength of cellulose and enhance the integrity of the plant cell walls.

In some embodiments, cell walls of a plant or plant waste is broken by using a physical process (e.g., a milling process and a pressure-releasing process), a chemical process (e.g., an acid treatment or base treatment), a biological process, or a combination thereof. In some embodiments, a pure mechanical process, a pure chemical process, or a pure biological process is used to break the cell walls. Once the cell wall is broken, the cell plasma including vitamins, proteins, antioxidants and all other nutrients mentioned above becomes further accessible to animals.

Cellulose is the most abundant material found in plants and has a lot of potential energy. However, not many animals can fully utilize it as a nutrient source. So far, there have been many microorganisms found to demonstrate cellulase activities. In some embodiments, the added microorganisms use cellulase to break down cellulose into glucose and use the glucose as a carbon source. In some embodiments, the microorganisms, enzymes, or substances that can break down the cellulose are added or used. The methods and devices disclosed herein are able to maximize nutrients and probiotics population of the products made (e.g., the animal feed).

Other features and advantages of the present invention will become apparent after reviewing the detailed description of the embodiments set forth below.

### BRIEF DESCRIPTION OF THE DRAWINGS

Embodiments will now be described by way of examples, with reference to the accompanying drawings which are meant to be exemplary and not limiting. For all figures mentioned herein, like numbered elements refer to like elements throughout.

FIG. 1 illustrates a system for converting agriculture waste in accordance with some embodiments.

FIG. 2 illustrates a method of converting plant waste into useful animal feed in accordance with some embodiments.

FIG. 3 illustrates a method of converting agriculture wastes to animal feed in accordance with some embodiments.

FIG. 4 illustrates a method of converting agriculture wastes to probiotic animal feed in accordance with some embodiments.

FIG. 5 illustrates a method of converting agriculture wastes to probiotic animal feed in accordance with some embodiments.

FIG. 6 illustrates a method of converting agriculture wastes to probiotic animal feed in accordance with some embodiments.

FIG. 6A illustrates a method of converting agriculture wastes to probiotic animal feed in accordance with some embodiments.

### DETAILED DESCRIPTION OF THE PREFERRED EMBODIMENT

Reference is made in detail to the embodiments of the present invention, examples of which are illustrated in the accompanying drawings. While the invention is described in conjunction with the embodiments below, it is understood that they are not intended to limit the invention to these embodiments and examples. On the contrary, the invention is intended to cover alternatives, modifications and equivalents, which can be included within the spirit and scope of the invention as defined by the appended claims. Furthermore, in the following detailed description of the present invention, numerous specific details are set forth in order to more fully illustrate the present invention. However, it is apparent to one of ordinary skill in the prior art having the benefit of this disclosure that the present invention can be practiced without these specific details. In other instances, well-known methods and procedures, components and processes have not been described in detail so as not to unnecessarily obscure aspects of the present invention. It is, of course, appreciated that in the development of any such actual implementation, numerous implementation-specific decisions must be made in order to achieve the developer's specific goals, such as compliance with application and business related constraints, and that these specific goals are vary from one implementation to another and from one developer to another. Moreover, it is appreciated that such a development effort can be complex and time-consuming, but is nevertheless a routine undertaking of engineering for those of ordinary skill in the art having the benefit of this disclosure.

Rather than recycling plant waste into the ground as compost, the present system and method create streams to further break down plant waste into three basic components: cellulose fiber, protein, and mineral. Cellulose fiber can be used as a raw material for alcohol production. In addition, the paper industry is able to use the cellulose fiber as bedding material for dairy cow and chicken farms.

Proteins, oils, organic antibiotics, vitamins, minerals, and antioxidants are also able to be recovered as animal feed. The inorganic salts, that are rich in potassium and phosphoric, are able to be recycled back into the system along with water to irrigate the land.

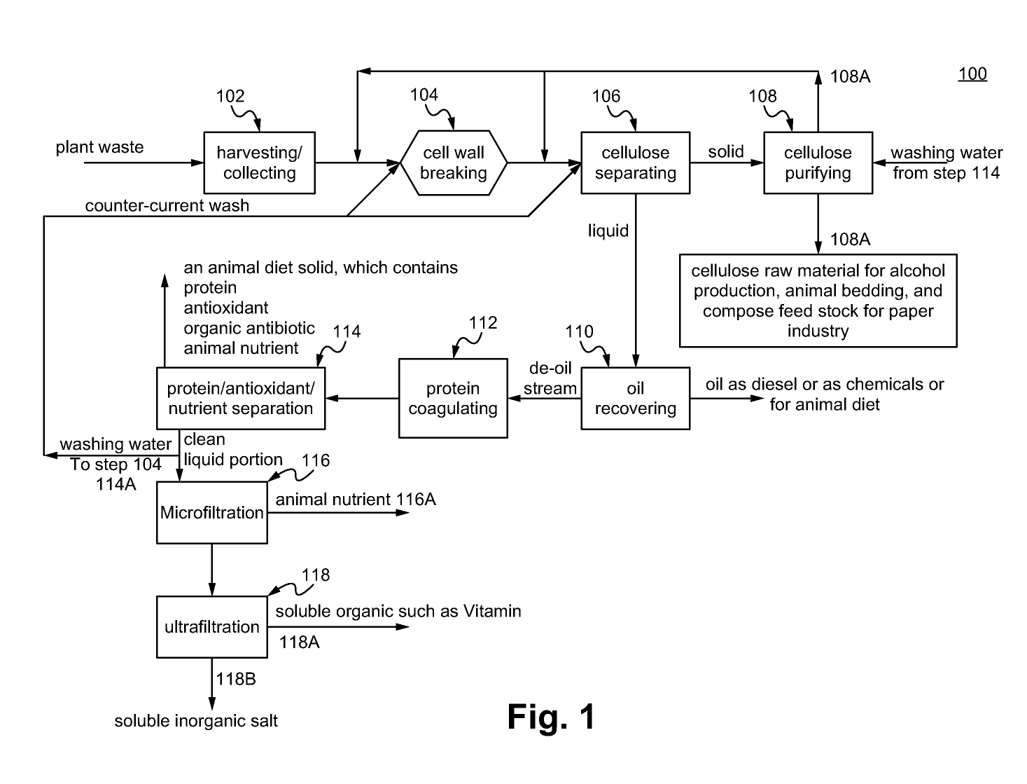

FIG. 1 illustrates a system 100 for converting agriculture waste in accordance with some embodiments. The system 100 is able to separate all of the raw components and make uses of them. The system 100 is able to be used in an industrial or commercial scale plant.

At Step 102, agricultural waste (such as plant waste, including roots, skins, seeds, stalks, leaves) and stems are harvested and collected. Added predetermined amount of water to the agriculture waste to have a 70 to 95% moisture content before feed to a cell wall breaking device. The water can be fresh water or the water/fluid stream from the Step 108. At Step 104, the slurry is fed to a cell wall breaking device to break cell walls of the agriculture waste, such that protein, oil, starch/sugar, antioxidant, nutrient material, and mineral from cellulose cell wall (mainly fiber) are released. In some embodiments, the cell wall breaking device is a milling device, such as hammer mill, disc mill, pin mill, disintegrator, and simple blender type milling devices. A person of ordinary skill in the art appreciates that any other milling/grinding devices are able to be used so long as the milling device is able to break the cell walls. In some embodiments, one or more discs type grinding mills are used. At Step 106, cellulosic separation is performed. Cellulose (fiber) is removed from the grinded mass slurry, such that the cellulose is separated from valuable products, such as protein, oil, antioxidant, and nutrient. In some embodiments, the cellulosic separation uses a screen separation device, such as a pressure screen, a paddle screen, a vibration screen, a screen centrifuge, and a press. A person of ordinary skill in the art appreciates that any other separation devices that are able to separate cellulose are within the scope of the present invention. In some embodiments, a screw press is used after using a paddle screen to have a higher protein recovery and maximum cellulose (fiber) cake dryness.

At Step 108, the cellulose removed from the Step 106 is purified. The purification process is able to be performed by adding washing water to the cellulose. In some embodiments, the washing water is able to be from Step 114 as a counter current water wash. In some embodiments, the water/fluid stream from the Step 108 is able to be added to the cell wall breaking Step 104 or to the cellulose separating Step 106 as counter current wash. The above described counter current washing and/or additional paddle screen are able to generate purer fiber and recover more valuable products, such as protein, oil and antioxidant. The cellulose isolated from the cellulose purification Step 108 is able to be collected at Step 108A as cellulose raw material, which is able to be used for alcohol production, animal feeding, and compose feed stock for paper industry. The fiber (cellulosic) is also able to be used as bedding material for dairy cow or chicken farm. With composting process, the cellulose is able to be used to produce organic fertilizer and soil condition materials. The cell wall that is broken by the grinding action speeds up the process of composting. The fiber is also able to be used as biodegradable oil absorber for oil spills, packing material, and other green technology.

At Step 110, oil recovering is able to be performed using the liquid from the cellulosic separation Step 106. The liquid from cellulosic separation Step 106 contains the entire valuable products, such as protein, oil, starch/sugar, antioxidant, nutrient, antibiotic, and hormone. When the agricultural waste used has very high oil content (such as orange peels), the oil recovering Step 110 is able to generate/recover significant amount of oil. At the Step 110, centrifuges such as decanter, desludger or disc decanter are able to be used. The oil recovered at the Step 110 is able to be used as fuel or for other industry usage. At Step 112, a de-oil stream from the oil recovering Step 110 is sent to the protein coagulating Step 112, such that the protein is able to be coagulated by heating (temperature higher than 160° F.) or by adjusting the pH (around 5 or in an acidic condition). At the Step 112, oil, starch, antioxidant, nutrient, antibiotic, and hormones absorb and coagulate with protein. At Step 114, a separation step is performed. The coagulated proteins with all the valuable elements in the plant wastes from the Step 112 are able to be separated from the liquid by using a simple separation/filtration step/device, such that proteins with valuable ingredients are recovered, which is able to be an ideal animal feed. The animal feed is deemed organic with minimum or without antibiotics and hormone addition. A clean liquid with all the minerals from the separating/filtrating Step 114 is able to be used as animal drinking water or as plant irrigation water. The liquid portion from the separating Step 114 is able to be used as a washing water 114A to be used at the Step 104. At Step 114, any liquid solid separation device such as bag filter, plate and frame filter, drum filter, centrifuges (decanter, basket, disc centrifuge and disc decanter etc)

At Step 116, a microfiltrating step is performed. The clean liquid from the Step 114 contains some soluble, valuable chemicals such as vitamins which can be further separated and recovered using a micron filtration device, such that animal nutrients are able to be isolated/recovered. At Step 118, ultra-filtrating process is able to be used, such that soluble organic substances 118A, such as vitamin, can be recovered, which are able to be used as an animal food. Soluble inorganic salt 118B from the ultrafiltrating Step 118 is able to be used as a plant food or inorganic chemicals.

In some embodiments, the cell wall breaking efficiency is able to be improved by applying heat, adjusting the pH value to around 8-10, or adding an enzyme or other chemicals before or during the cell wall breaking Step 104.

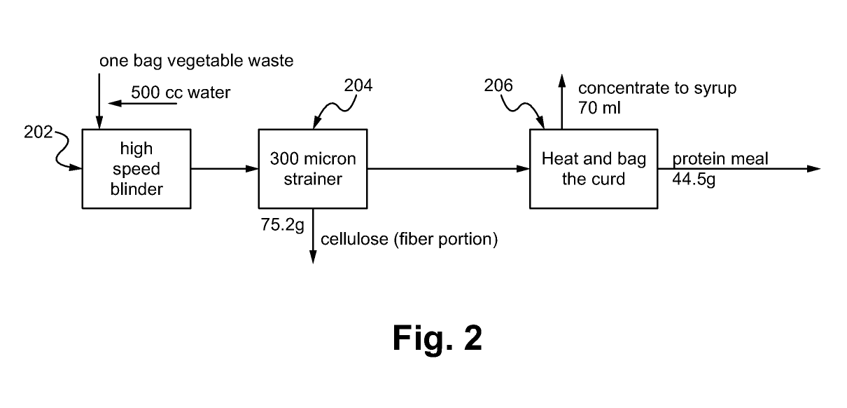

FIG. 2 illustrates a method of converting plant waste to useful animal feed in accordance with some embodiments. At Step 202, 500 c.c. of water is mixed with a grocery shopping bag amount of Swiss chard (600 g) in a 2,000 c.c. high speed kitchen blender. The bag of Swiss chard and water are grinded for 4 minutes. The grinded slurry is poured into a 300 micron kitchen strainer 204. The fiber portion is on the top portion/layer of the strainer. The grinded slurry is lightly pressed to remove as much liquid as possible. At Step 206, the liquid from the kitchen strainer 204 is collected and heated to around 180° F. The heating causes the proteins to coagulate and form a curd. This curd slurry was poured onto a cloth separating the curd from clean liquid. The curd was lightly pressed to produce a protein cake which contained all the antioxidants (green color) and nutrients. Both protein cake and fiber portion are sun dried. A person of ordinary skill in the art appreciates that any other drying processes are able to be used. After the drying process, the fiber portion and the protein portion are weighed and analyzed. The clean liquid from the Step 206 is dried (e.g., dried under sun) to remove most of the water producing a concentrated syrup, which is placed in a refrigerator overnight. The inorganic salt is crystallized out and settled in bottom. The weight of this syrup is analyzed.

The fiber, protein and syrup from one bag of Swiss chard is weighed and analyzed for protein, oil, starch, ash, nutrient and net energy, which is able to be used as animal feed. The analytic results of the components are shown in Table 1.

TABLE 1

| | | | | | Vegetable |
|---|---|---|---|---|---|
| | unit | Protein | Mineral | Fiber | Waste |
| weight produce | gram | 44.5 | 71 | 75.2 | % in dry |
| product | | | | | base |
| % moisture in | % | 12.99 | 61.47 | 9.84 | |
| product | | | | | |
| dry weight product | gram | 38.72 | 27.36 | 67.80 | 133.88 |
| % dry weight | % | 28.92 | 20.43 | 50.64 | |
| distribution | | | | | |
| protein (crude) | % | 35.7 | 10.1 | 10.3 | 17.60528 |
| Fat(crude) | % | 6.3 | 1.58 | 1.22 | 2.762793 |
| Fiber (acid | % | 22.3 | 0.01 | 22.3 | 17.74525 |
| detergent) | | | | | |
| ash | % | 10 | 34 | 12.8 | 16.32221 |
| Total digestible | % | 74.7 | 59.4 | 70.9 | |
| nutrients | | | | | |
| Net energy | Mcal/ | 0.78 | 0.61 | 0.73 | |
| (lactation) | Lbs. | | | | |
| Net energy(maint.) | Mcal/ | 0.79 | 0.59 | 0.74 | |
| | Lbs. | | | | |
| Net energy (gain) | Mcal/ | 0.54 | 0.36 | 0.49 | |
| | Lbs. | | | | |
| Digestible energy | Mcal/ | 1.49 | 1.19 | 1.42 | |
| | Lbs. | | | | |
| Metabolizable | Mcal/ | 1.33 | 1.12 | 1.38 | |
| energy | Lbs. | | | | |
| Starch (total) | % | 4.61 | 9.06 | 3.06 | |
| weight of protein | gram | 13.82 | 2.76 | 6.98 | 23.57 |
| weight of oil | gram | 2.44 | 0.43 | 0.83 | 3.70 |
| weight of fiber | gram | 8.63 | 0.00 | 15.12 | 23.76 |
| weight of starch | gram | 1.78 | 2.48 | 2.07 | 6.34 |
| weight of ash | gram | 3.87 | 9.30 | 8.68 | 21.85 |
| % protein | % | 58.65 | 11.72 | 29.63 | 100.00 |
| distribution | | | | | |
| % oil distribution | % | 65.95 | 11.69 | 22.36 | 100.00 |
| % fiber distribution | % | 36.35 | 0.01 | 63.64 | 100.00 |
| % starch distribution | % | 28.16 | 39.10 | 32.73 | 100.00 |
| % ash distribution | % | 17.72 | 42.57 | 39.72 | 100.00 |

The data show that the protein portion (e.g., the solid portion from the Step 114) has 35.7% protein, 6.3% oil and 22% fiber, which is ideal to be used as a chicken diet or pig diet. The combination of the cell breaking process and the protein recovering process is able to produce a protein meal as animal feed. The protein meal is able to have 58.65% protein recovery, 65.95% oil recovery, and only content 36.36% of total fiber in plant waste. These fine fibers produced using an embodiment of the present invention are finer and hemicellulose, which are able to be consumed and easily converted to energy by chickens and pigs. This protein product has 1.49 Kcal/lb. of digestible energy and 1.33 Kcal/lb. of metabolized energy.

In the following, Table 2 shows a composition table of some selected agriculture wastes that is able to be used in accordance with some embodiments.

TABLE 2
Composition of agriculture wastes

| | | | | | Nitrogen- | | |
|---|---|---|---|---|---|---|---|
| | Crude | Crude | Ether | Ash | free | | Crude |
| | protein | fiber | extract | content | extract | Crude fat | ash |
| Vegetable leave | 14.85% | 18.21% | | 15.21% | | | |
| waste | | | | | | | |
| Straw | 3.25% | 44.30% | 0.85% | 20.75% | 30.85% | | |
| Husk rice bran | 15.50% | 8.50% | 1% | 8% | | | |
| Corn stalk | 6.20% | 23.40% | | | 62.90% | 2.30% | 5.30% |
| Corn cobs | 4.20% | 23.80% | | | 67.40% | 2.80% | 1.80% |
| Soybean vine | 11.50% | 22.31% | | | | | |
| Wheat bran | 14.10% | 10.10% | | | | 3.90% | |
| Sugar cane | 4.50% | | | | | | |
| leaves | | | | | | | |

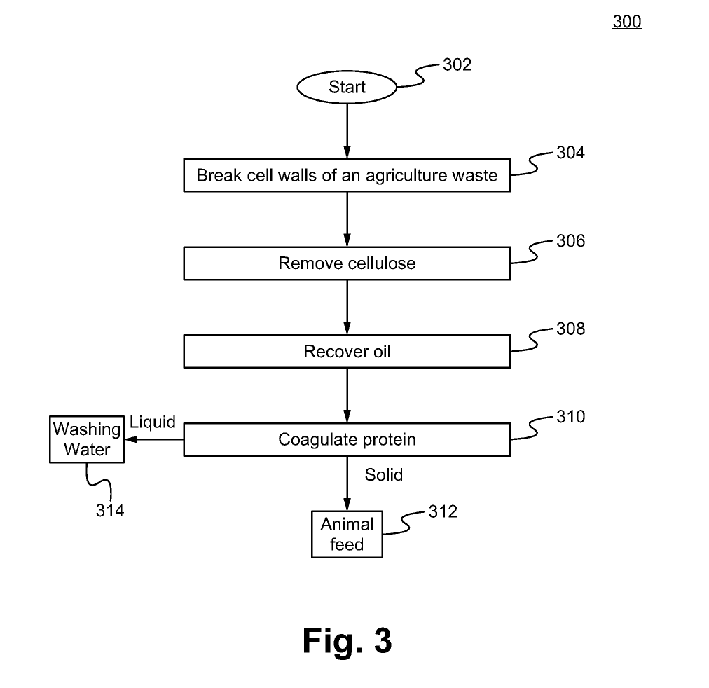

FIG. 3 illustrates a method 300 of converting agriculture wastes to animal feed in accordance with some embodiments. The method 300 is able to start at Step 302. At Step 304, cell walls of an agriculture waste in a slurry form are broken. At Step 306, cellulose is removed from the slurry. At Step 308, oil is recovered. At Step 310, proteins are coagulated. The liquid portion from the Step 310 is able to be used as washing water at Step 314. The solid portion from the Step 310 is able to be used as animal feed at Step 312.

Artificial antibiotics are heavily used in animal diet, which becomes a serious health issue for humans that consume these animals. Solutions to the issue stated above are needed. Plants, vegetables, and fruits possess high sources of vitamins and minerals which are ideal sources for making free of artificial antibiotic animal feed. The animal feed produced by the process and system in accordance with some embodiments is rich in fine/soluble fiber, proteins, hydrocarbon, oils, organic antibiotics minerals, and antioxidants, which is ideal for pure organic diet for animals.

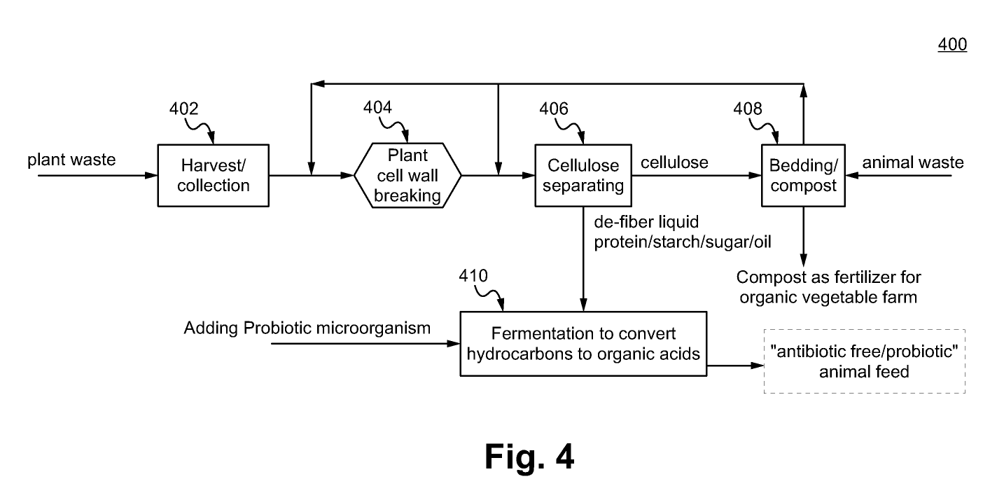

FIG. 4 illustrates a method 400 of converting agricultural wastes to animal feed in accordance with some embodiments. At a Step 402, one or more plant wastes go through a harvest/collection process.

At a Step 404, breaking plant cell wall is performed. In some embodiments, the breaking of the plant cell wall is performed by using a milling device. In some embodiments, the breaking of the plant cell wall uses a pure mechanical force. In some embodiments, the milling device comprises a hammer mill, a disc mill, a pin mill, a disintegrator, a simple blender type mill, or a combination thereof.

At a Step 406, separating fiber/cellulose is performed, which forms two portions. A first portion comprises fibers and celluloses and a second portion comprise slurry as a liquid portion. Fibers and celluloses are separated from valuable nutrients/substances in the slurry. The slurry is rich in hydrocarbons (sugar, starch and fine fiber), oil, proteins, minerals, and vitamins. The separated fiber and cellulose portion are able to be used as animal bedding.

At a Step 408, the portion of the separated fiber and cellulose from the Step 406 are able to be combined with an animal waste to form an organic fertilizer, which is able to be used by an organic farm. The liquid portion from the Step 406 containing valuable ingredients for animal is able to be further enriched by a fermentation process.

At a Step 410, fermenting is performed. Microorganisms, enzymes of the microorganisms, and/or enzymes are added to the container performing the fermenting process. In some embodiments, the fermentation is performed/accomplished in a slightly acidic to a neutral environment, such as pH 5.5 to pH 7. Temperature is able to be between 25° C. to 40° C. depending on the properties of microorganisms and enzymes used during this step to maximize the growth rate of selected microorganisms and conversion rate of selected enzymes. In some embodiments, the amount of microorganisms added varies because of different growth rate of different microorganisms (e.g., 100 proportion of the fermentation volume to 1 proportion of the nearly-saturated microorganism culture, normally around 1×10⁹ CFU/mL). The added microorganisms, enzymes of the microorganisms, and/or enzymes covert hydrocarbon(s) to organic acid and propagate more good bacterial to generate probiotic food, which is used as “antibiotic free animal feed.”

In some embodiments, the microorganisms used as probiotics at the Step 410 include Bacillus sp., Lactobacillus sp., Bifidobacterium sp., Saccharomyces cerevisiae and/or Monascus purpureus. These microorganisms are able to be natural strains or engineered strains with enhanced abilities including stronger environmental tolerances, higher growing speed, higher enzyme production efficiency, stronger enzyme stabilities, and/or higher enzyme kinetics.

In some embodiments, the microorganisms are added externally. In some other embodiments, the microorganisms are originally existed and an environment condition suitable to enhance the activity or facilitate the growth of the microorganisms is optimized and provided. In some embodiments, the microorganisms described herein includes the probiotic microorganisms.

In addition, the microorganisms are able to be used individually or in any combination to meet target requirements for different kinds of animal and for different ages. For example, Bacillus thuringiensis is able to undergo co-fermentation with Monascus purpureus to produce red yeast rice for animals and minimize fly population locally at farm at the same time.

Similarly, engineered Bacillus subtilis exhibiting higher cellulase kinetics can undergo co-fermentation with engineered Lactobacillus acidophilus, which generates a higher lactic acid production rate to maximize conversion efficiency.

Further, the engineered protease demonstrating higher kinectics and heat resistance is able to be added with Lactobacillus delbrueckii subsp. bulgaricus, which is able to break down protein to amino acids making the products more accessible to animals and maximizing the probiotic population for piglets.

In some embodiments, various kinds of agricultural wastes produced by farms during harvest are used including (1) stalks and straws, (2) hull and cobs, 3) vine leaf, pod and tub, and 4) old palm and banana plant. Crop wastes produced by farms are several times higher in quantity when compared with the actual crop produced. The crop wastes that have relatively low protein content (3 to 12% protein) have less nutrient value. As a result, it has a very limited use as animal feed (mainly for cows and horses). It is normally placed underground as soil conditioner and fertilizer. Recycling agricultural waste is a sustainable manner, which avoids current burning methods that create unhealthy air pollution.

In some embodiments, the method 400 is able to be similar to the method of FIG. 1. In some embodiments, the plant waste at the Step 402 of method 400 contains a high percentage of hydrocarbons and a low percentage of oil and protein. In such case, the oil and protein recovery steps, as described in the method 100 of FIG. 1, is able to be skipped and directly move to the fermenting of the Step 410, such that hydrocarbons are converted to organic acids.

In some embodiments, the organic acids in animal feeds include lactic acid, proprionic acid, benzoic acid, and butyric acid. In some embodiments, the organic acids are generated in the fermenting process. In some embodiments, the organic acids are added externally. These organic acids lower the pH value during fermentation step and make the overall conditions difficult for other bacteria to grow, which indirectly prevent contamination. In addition, these organic acids can increase feed intake and facilitate digestion. The active, nonpathogenic microorganisms (e.g., probiotics) can compete binding sites and nutrients with potentially pathogenic microorganisms, which minimize the population of bad microorganisms. In addition, most probiotics have been proven capable of enhancing the host's immune system and suppress inflammatory reactions, which also help host to fight potential pathogens. With these advantages, probiotics are able to be used to reduce or avoid the use of the antibiotics in animal feed.

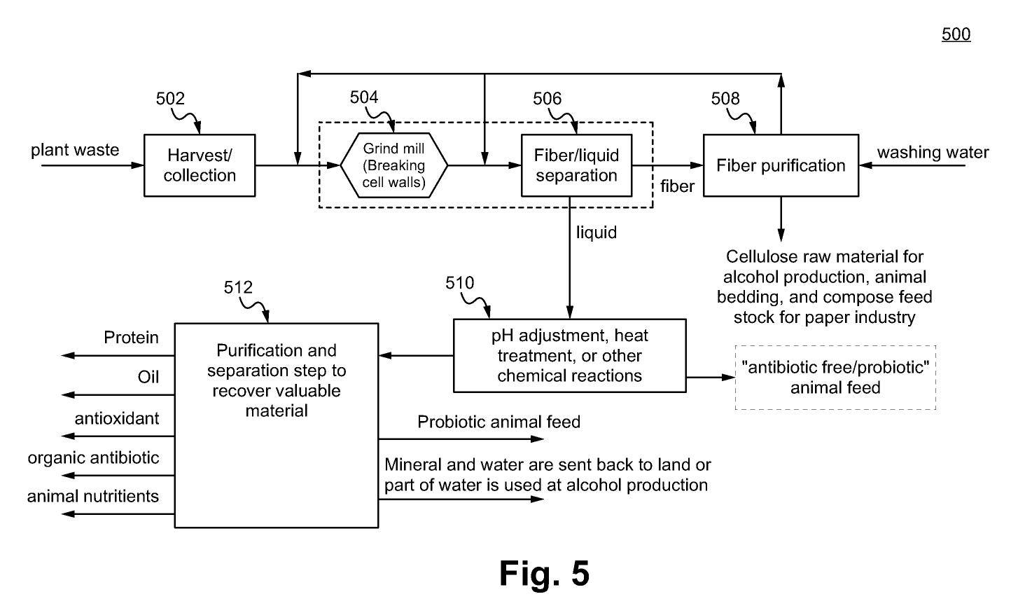

FIG. 5 illustrates a method 500 of converting agricultural wastes to animal feed and produce alcohol in accordance with some embodiments. At a Step 502, the plant wastes go through harvest/collection.

At a Step 504, the plant waste from the Step 502 is sent to a cell breaking process, which is able to be done using a pure mechanical force, such as using a milling device.

At a Step 506, a liquid/solid separation process is performed, which separates and forms a fiber portion (cellulose) and a liquid portion (valuable nutrient for animal), such that the cellulose is separated from the liquid portion. In some embodiments, the fiber portion is sent to a purifying process 508. The fibers from the Step 508 is able to be purified for alcohol production. The liquid portion from the Step 506 with valuable animal nutrient is able to be used directly as part of animal diet. In some embodiments, the liquid portion from the Step 506 goes through various processes to recover/extract valuable ingredients including oil, protein, antioxidant, mineral, vitamin, and probiotic feed for animal. In some embodiments, the liquid portion from the Step 506 is sent to a Step 510.

At the Step 510, the liquid portion is treated by pH adjustment, heat treatment, and/or adding chemicals. In some embodiments, the adjustment of pH can be performed by adding one or more of the bases and/or acids (e.g., organic and/or inorganic bases/acids). By gradually increasing or decreasing pH, some proteins and nutrients precipitate to the bottom of the liquid for easier separation. In some embodiments, the final pH range is slightly acidic to neutral, pH 5.5 to pH 7. In some embodiments, various salts can be added to precipitate various chemicals, including antioxidants, in the liquid. In some embodiments, temperature is set between 25° C. and 60° C. depending on different reactions. At a Step 512, purifying and separating are performed to isolate/separate individual ingredients.

In some embodiments, various additional processes are able to be added or removed from the above disclosed methods while the processes of cell breaking (e.g., the Step 504) and liquid/solid separating (e.g., the Step 506) are performed.

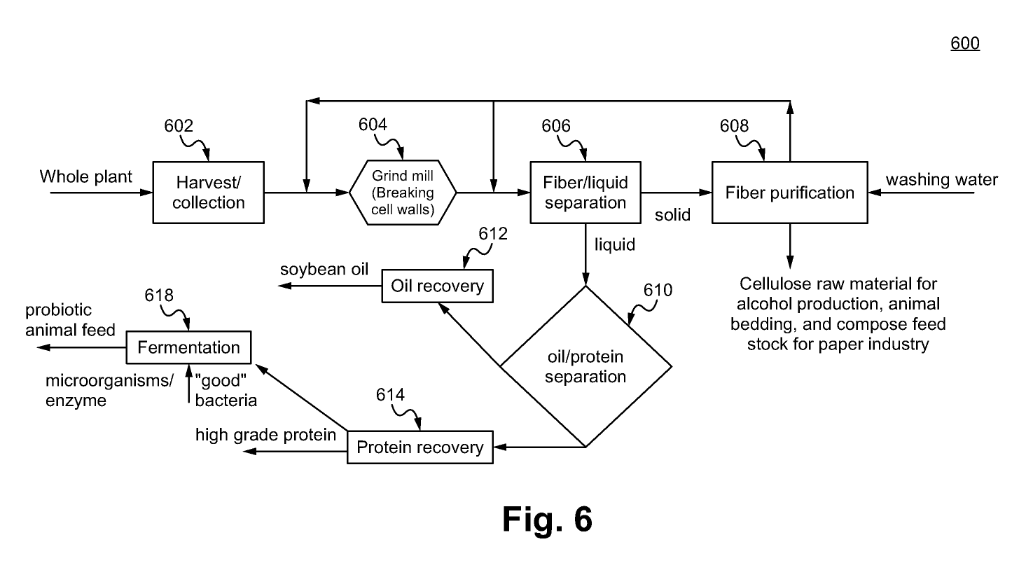

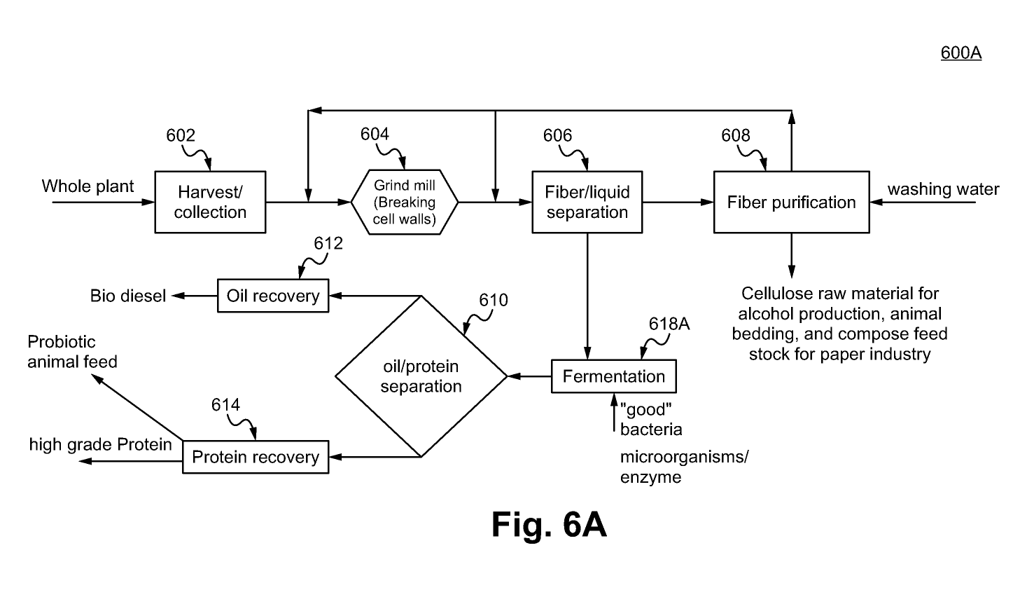

FIG. 6 illustrates a probiotic animal feed producing method 600 in accordance with some embodiments.

At a Step 602, the plant wastes (e.g., vine and pod) or whole plant are harvested/collected. In some embodiments, the plant waste comprises soybeans and/or corns. Soybeans and corns are two major crops produced in the U.S. The soybean plant waste (vine and pod) or whole soybean plant (including soybeans) have more than 12% of protein, which is a nutrient that animals need. The oil, protein antioxidant, and nutrients are extracted and recovered to make probiotic animal feed. In some embodiments, the valuable ingredients are extracted/recovered from the plant wastes before converting fiber to alcohol.

At a Step 604, a cell breaking process is performed on the plant wastes from the Step 602. The cell walls of the plant waste are ruptured and broken using one or more breaking devices, such as a grinding mill. The grinding mill is able to be configured to have a force or grinding condition sufficient to break not only the shell of the plants but also the cell walls of the plant wastes. In some embodiments, all or substantially all of the plant wastes having majority of the cell walls are broken. In other embodiments, more than half of the cell walls of the plant wastes having at least one opening with a hole size sufficient for the majority of the preselected nutrients to come out. In some embodiments, the milling device is configured to perform a two stage milling, including breaking the shell of the plant waste at a first stage and breaking the cell walls of the plant waste at a second stage.

At a Step 606, a solid and liquid separating process is performed, such that the solid portion (fiber portion) is separated from the liquid portion (valuable nutrient feed ingredient).

At a Step 608, a purifying process is performed. The solid portion from the Step 606 go through the purifying process of the Step 608 before the substance is sent to an alcohol production process.

At a Step 610, an oil/protein separating process is performed. The liquid portion from the Step 606 is sent to a separation device (e.g., centrifuges including a nozzle centrifuge, Desludger centrifuge, a disc decanter, or a three phases decanter) at the Step 610, such that the oil is separated from the protein by density.

At a Step 612, an oil recovering process is performed. The light phase (oil phase) from the separation device of the Step 610 is sent to oil recovering device at the Step 612 to produce oil (e.g., soybean oil).

At a Step 614, a protein recovering process is performed. The heavy phase from the separation device of the Step 610 is sent to a protein recovery device at the Step 614 to produce protein (e.g., soybean protein).

At a Step 618, a fermenting process is performed. One stream (e.g., the remainder substance) from the Step 614 is sent to the fermentation device at the Step 618 to produce probiotic animal feed.

FIG. 6A illustrates another probiotic animal feed producing method 600A in accordance with some embodiments. The method 600A comprises processes similar to the processes of method 600 (FIG. 6). The process steps described in the method 600 are applicable to the process steps of the method 600A. In some embodiments, a fermenting process at a Step 618A is performed, which is able to be a process performed between the Step 606 (fiber/liquid separation) and the Step 610 (oil/protein separation). At the Step 618A, the organic acid (such as lactic acid) and enzyme that are produced through the fermenting at the Step 618A is able to improve the separation efficiency of the Steps 610, 612, and 614 (oil/protein separation, oil recovering, and the protein recovering).

In some embodiments, probiotics refer to microorganisms that provide health benefits when consumed by humans or animals. The term probiotic is able to refer to ingested microorganisms associated with benefits for humans and animals. The benefits of probiotics include the decrease of potentially pathogenic gastrointestinal microorganisms, the reduction of gastrointestinal discomfort, the strengthening of the immune system, the improvement of the skin's function, the improvement of bowel regularity, the strengthening of the resistance to cedar pollen allergens, the reduction or prevention of body pathogens, the reduction of flatulence and bloating, the protection of DNA, the protection of proteins and lipids from oxidative damage, and the maintaining of individual intestinal microbiota in subjects receiving antibiotic treatment. In some embodiments, the probiotic animal feed is able to be used to feed the animals, such that antibiotics do not need to be used in treating and preventing the bacterial infections during the growth of the animals.

In some embodiments, microorganisms, enzymes of the microorganisms, and/or enzymes are generally referred as “the probiotic substances.” The environment suitable for the growth/enhancement of the probiotic substances are generally referred as “the probiotic environment.”

To utilize, the probiotic substances are added or the probiotic environment are provided to facilitate the conversion of the hydrocarbon(s) to organic acid and propagate more good bacteria to generate probiotic food (e.g., antibiotic free animal feed.)

In operation, the wastes are collected, the cell walls of the plant wastes are broken to release the proteins and nutrients along with other useful components. The probiotic substances are added or the probiotic environment is provided at the fermentation, such that a probiotic animal feed is produced.

The present invention has been described in terms of specific embodiments incorporating details to facilitate the understanding of principles of construction and operation of the invention. Such reference herein to specific embodiments and details thereof is not intended to limit the scope of the claims appended hereto. It is readily apparent to one skilled in the art that other various modifications can be made in the embodiment chosen for illustration without departing from the spirit and scope of the invention as defined by the claims.

What is claimed is:

1. A method of making probiotic animal feed comprising:
a) adding one or more probiotic substances to a fermenting; and
b) generating a probiotic animal feed.

2. The method of claim 1, further comprising breaking cell wall of an agriculture substance before fermenting.

3. The method of claim 1, further comprising separating a liquid portion from a solid portion, wherein the liquid portion is sent to the fermenting.

4. The method of claim 1, wherein the probiotic substances comprise microorganisms.

5. The method of claim 1, wherein the probiotic substances comprise enzymes of microorganisms.

6. The method of claim 1, wherein the probiotic substances comprise enzymes.

7. The method of claim 1, further comprising converting hydrocarbons to organic acids.

8. The method of claim 1, further comprising co-fermenting with an engineered Bacillus subtilis and engineered Lactobacillus acidophilus.

9. A method of making probiotic animal feed comprising:
a) providing an environment suitable for the proliferation of probiotic microorganisms; and
b) generating a probiotic animal feed.

10. The method of claim 9, further comprising increasing the number of probiotic microorganisms.

11. The method of claim 9, further comprising a fiber and liquid separating, which generates a fiber portion and a liquid portion.

12. The method of claim 11, further comprising adjusting a pH value of the liquid portion to a range between 5.5 and 7.

13. The method of claim 11, further comprising precipitating proteins and nutrients by performing the pH adjusting.

14. A method of making probiotic animal feed comprising:
a) collecting an agriculture substance;
b) breaking cell walls of the agriculture substance;
c) separating a liquid portion from a solid portion;
d) adding microorganisms or enzymes to the liquid portion; and
e) forming a probiotic animal feed.

15. The method of claim 14, further comprising fermenting the liquid portion.

16. The method of claim 14, further comprising adjusting a pH value of the liquid portion.

17. The method of claim 14, further comprising recovering proteins, oil, antioxidants, organic antibiotics, animal nutrients, or a combination thereof.

18. The method of claim 14, further comprising separating oil and protein in the liquid portion.

19. The method of claim 18, further comprising fermenting after the separating oil and protein in the liquid portion.

20. The method of claim 19, further comprising recovering protein between the fermenting and the separating oil and protein in the liquid portion.

22. The method of claim 18, further comprising fermenting between the separating a liquid portion from a solid portion and the separating oil and protein in the liquid portion.

23. A probiotic animal feed making system comprising:
a) a cell wall breaking device;
b) a fermentor; and
c) an amount of probiotic substances in the fermentor.

24. The system of claim 23, wherein the probiotic substances comprise an amount of added microorganisms.

25. The system of claim 23, wherein the probiotic substances comprise bioengineered microorganisms.

26. The system of claim 23, wherein the probiotic substances comprise natural microorganisms.

27. The system of claim 23, wherein the probiotic substance comprises enzymes.

28. The system of claim 23, further comprising organic acids in the fermentor.

29. The system of claim 28, wherein the organic acids are at a level sufficient to inhibit bacteria that are harmful to animal, human, or both.

30. The system of claim 28, wherein the organic acids comprise lactic acid, proprionic acid, benzoic acid, butyric acid, or a combination thereof.

31. The system of claim 28, wherein the organic acids forms organic anti-microbial substances.
